(19) United States
(12) Patent Application Publication
(10) Pub. No.: US 2004/0072124 A1
(43) Pub. Date: Apr. 15, 2004

(54) Endodontic files made using bulk metallic glasses

(76) Inventors: Michael J. Kaufman, Gainesville, FL (US); W. Gregory Sawyer, Gainesville, FL (US)

Correspondence Address: Dennis P. Clarke, Miles & Stockbridge P.C., Suite 500, 1751 Pinnacle Drive, McLean, VA, 22102-3833, US

(21) Appl. No.: 10/457,014

(22) Filed: Dec. 12, 2003

Related U.S. Application Data
(60) Provisional application No. 60/386,455, filed on Jun. 7, 2002.

(51) Int. Cl. A61F 2/28; A61C 5/02; A61C 8/00; B65D 83/10

(52) U.S. Cl. 433/102; 623/23.53; 433/201.1; 206/369

(57) ABSTRACT

A medical implant or device comprising at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, the alloy comprising an amorphous phase as a volume fraction of at least 50%.

### BACKGROUND OF THE INVENTION

Previously known endodontic root canal files have been primarily made from carbon steel or stainless steel wire blanks which are ground to a desired size, taper and cross-sectional shape (for example, square, triangular or rhomboid). The wire blank is gripped on a first end while spring-loaded jaws secure the ground portion of the blank. The blank is then rotated from the gripped end while the jaws are moved axially away from that end. The jaws which secure the ground portion move along the ground wire blank, but do not allow the distal end of the blank to twist, thereby forming a twisted portion and helical flutes from the edges of the blank between the gripped end and the jaws. The cross-sectional shape, size and taper as well as the speed of twisting and spring force may be controlled to attain the desired properties in the final product. One such endodontic instrument is shown in U.S. Pat. No. 4,443,193.

With the introduction of superelastic materials such as nickel titanium alloys, it has been recognized that superelastic endodontic files would provide more elastic flexibility in bending and torsion than the previous steel files. The paper, An Initial Investigation of the Bending and Torsional Properties of Nitinol Root Canal Files, Walia et al., Journal of Endodontics, Volume XIV, No. 7, July 1988, studied the feasibility of manufacturing superelastic nickel titanium root canal files and evaluated the bending and torsional properties of these instruments.

In order to provide an endodontic file with a low modulus of elasticity which is more flexible in bending and torsion than conventional steel files and to overcome the problems with grinding disclosed in the Walia article and which the U.S. Pat. Nos. 5,464,362, 5,527,205 and 5,628,674 patents continue to have, a preground superelastic blank of a predetermined cross-sectional shape is twisted to provide helical flutes.

Superelastic materials are alloys which return to their original shape after substantial deformation. Superelastic alloys such as nickel titanium (NiTi) can withstand several times more strain than conventional materials, such as stainless steel, without becoming plastically deformed. Further, a superelastic material will generally recover approximately 6% after twisting at ambient temperature while a stainless steel will recover only 1-2% after twisting. Typically, superelastic alloys undergo a stress induced martensitic transformation which allows for shape memory properties. Shape memory and superelasticity are found in stoichiometric NiTi, near-equiatomic Ni—Ti, for example, 50.8 atomic percent Ti and 49.2 atomic percent Ni, Ni—Ti—Cu, Ni—Ti—Nb and Ni—Ti—Fe alloys as well as other alloys. Examples of suitable nickel-titanium alloys in various stoichiometric ratios are disclosed in U.S. Pat. No. 5,044,947 (nickel-titanium-copper alloy) and U.S. patent applications Ser. Nos. 08/221,638 and 08/454,016, inventor Sachdeva et al., entitled “NiTiNb Alloy Processing Method and Articles Formed Thereby” (nickel-titanium-niobium-alloy). Nitinol is a commonly used Ti—Ni alloy with shape memory behavior that is used in many types of medical device applications.

Other examples of shape memory alloys include those described in U.S. Pat. Nos. 4,665,906 and 5,067,957 which describe medical devices and methods of installation using a non-specific shape memory alloy which displays stress induced martenistic behavior, versus an activation temperature.

PCTAUS 96/00016 (Pub. No. 96/38097) by Farzin-Nia and Sachdeva describes a dental or orthodontic article comprising an alloy having a primary constituent of at least one of the group consisting of Ti, Zr, Si, Mo, Co, Nb, and Be; and at least one secondary element selected from the group consisting of Ta, Cu, Al, V, Pd, Hf and Fe, and where the primary constituent is in the range of about 30 to 85 percent by weight. Subsequent claims specify the preference for a Ti or Zr base alloy.

Other patents refer to metal alloy compositions for endodontic dental files. U.S. Pat. No. 5,380,200 relates to a bi-metallic dental file with a flexible core comprising NiTi alloy, stainless steel, or any Ti alloy.

U.S. Pat. Nos. 5,655,950; 5,628,674; 5,527,205 and 5,464,362 describe the machining and grinding method for a dental file made of a metallic material comprised of at least 40 percent titanium and which has a diameter less than about 0.07 inches.

A more recent patent, U.S. Pat. No. 4,857,269, also deals with the desirability of low elastic modules in medical devices. This patent describes a titanium based alloy (versus Nb-base) consisting of an amount of up to 24 weight percent of isomorphous beta stabilizers Mo, Ta, Nb and Zr, providing that the molybdenum, if present, is at least 10 weight percent, and when present with zirconium, is between 10 and 13 weight percent with the zirconium being between 5 and 7 weight percent. Additionally, the same titanium based alloy also has up to 3 weight percent eutectoid beta stabilizers selected from Fe, Mn, Cr, Co and Ni, wherein the combined amount of isomorphous and eutectoid beta stabilizers is at least 1.2 weight percent. Optionally, up to 3 weight percent aluminum and lanthanum can be present in the alloy with the elastic modulus not exceeding 100 GPa (14.5 MSI). Examples include Ti-10-20Nb-1-4Z-2Fe-0.5Al (TMZF.TM.).

In an effort to improve both the bio-compatibility and to reduce elastic modulus in a titanium alloy, Davidson and Kovacs (U.S. Pat. No. 5,169,597) developed a medical implant titanium alloy with 10-20 weight percent Nb, or 30-50 weight percent Nb and 13-20 weight percent Zr, or sufficient Nb and/or Zr to act as a beta stabilizer by slowing transformation of beta (U.S. Pat. No. 5,545,227), where toxic elements are excluded from the alloy. The preferred example is Ti-13Nb-13Zr (Ti 1313.TM.). Tantalum can also be used in the '227 patent as a replacement for niobium where the sum of Nb and Ta is 10-20 weight percent of the alloy. All of these patents describe the use of Ti, Nb, and/or Zr. Others such as I. A. Okazaki, T. Tateishi and Y. Ito, have also proposed Ti-based alloy compositions including Ti-15Zr-4Nb-2Ta-0.2Pd and variations of the type Ti-5Zr-8Nb-2Ta-10-15-Zr-4-8-Nb-2-4 Ta, Ti-10-20Sn-4-8Nb—Ta-0.2Pd, and Ti-10-20 Zr4-8Nb-0.2Pd.

The entire disclosures and contents of each of the above-discussed patents are incorporated herein by reference.

### SUMMARY OF THE INVENTION

An embodiment of the invention relates to a medical implant or device comprising at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, the alloy comprising an amorphous phase having a volume fraction of at least 50%.

A second embodiment of the invention relates to a kit comprising a plurality of the medical implants and/or devices described above.

A third embodiment of the invention relates to a method of manufacturing a medical implant or device comprising:

(a) providing a precursor blank;

(b) forming the precursor blank into a medical implant or device that is sized for its intended use, the precursor blank comprising at least one component at least partially fabricated from an alloy described above.

Other embodiments of the invention concern uses of the medical implants and/or devices and kits described above.

Additional embodiments of the invention relate to articles of manufacture comprising packaging materials and medical implants, devices or kits as described above wherein the packaging material comprises a label which indicates that the medical implant, device or kit can be used for the uses described herein.

### DETAILED DESCRIPTION OF THE INVENTION

The present invention is predicated on the discovery that the recently developed bulk metallic glasses (also known as bulk amorphous alloys or liquid metal alloys) are ideally suited for the construction of medical devices and implants, in particular, endodontic instruments, most preferably, endodontic files. Endodontic files are currently made from stainless steel or nitinol. Dentists usually prefer the nitinol-based files because nitinol is more flexible than stainless steel with similar tool life characteristics. However, nitinol is considerably more expensive than stainless steel.

The bulk amorphous alloys have very high strength which correlates with good edge retention and tool life characteristics and low elastic moduli in knives constructed therefrom making them very flexible. Furthermore, unlike nitinol, the processing strategies for these types of alloys are considerably easier making them suitable for large-scale production of medical devices and implants at economical prices.

The alloys suitable for use in constructing the implants and devices of the invention as well as methods for their production and processing are described in U.S. Pat. Nos. 5,032,196; 6,450,696 and 6,027,586.

Thus, endodontal files can be cast or formed readily using the known processing methods described in the above-noted patents and the resulting files will be usable immediately after casting. The current stainless steel and nitinol files must be machined and this process adds considerable cost to each part even though the technology for machining them is fairly mature. Thus, the files of the invention can be produced much less expensively than those currently in use. Files currently available commercially can be used to create a pattern from which the files of the invention can be cast or formed from the bulk metallic glass into the same shapes.

Dentists doing root canals will greatly appreciate these highly flexible and wear resistant files.

Alloys having a composition represented by the general formula Xₐ M_{b} Al_{c}, where X is Zr or Hf, M is at least one element selected from the group consisting of Mn, Fe, Co, Ni, Ti and Cu, and a, b and c are, in the atomic percentages, 25<a<85, 5<b<70 and 0<c<35, and comprises an amorphous phase as a volume fraction of at least 50% may be employed in the practice of the invention. Specific compositions include amorphous alloys each having a composition represented by Zr₆₀Al₁₅Co_{2.5}Ni_{7.5}Cu₁₅ or Zr₆₅Al₁₀Ni₁₀Cu₁₅ (where the subscript represents the atomic percentage of each element).

The entire contents and disclosures of each and all U.S. patents cited herein are expressly incorporated herein by reference.

1. A medical implant or device comprising at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, said alloy comprising an amorphous phase as a volume fraction of at least 50%.

2. A medical implant or device of claim 1 wherein said alloy has a composition represented by the formula, Zr₆₀Al₁₅Co_{2.5}Ni_{7.5}Cu₁₅.

3. A medical implant or device of claim 1 wherein said alloy has a composition represented by the formula, Zr₆₅Al₁₀Ni₁₀Cu₁₅.

4. A medical implant or device of claim 1, wherein the medical implant or device is an orthodontic, endodontic, or dental implant or device.

5. A medical device of claim 4, when the device is shaped as an endodontic instrument which is adapted for use in performing root canal therapy.

6. A medical device of claim 5, when the device is shaped as an endodontic file configured for root canal insertion.

7. A kit comprising a plurality of medical implants and/or devices of claim 1.

8. A kit comprising a plurality of medical implants and/or devices of claim 4.

9. A kit comprising a plurality of endodontic instruments according to claim 5.

10. A kit comprising a plurality of endodontic files according to claim 6.

11. A method of manufacturing a medical implant or device comprising:
(a) providing a precursor blank;
(b) forming the precursor blank into a medical implant or device that is sized for its intended use, the precursor blank comprising at least one component at least partially fabricated from an alloy of claim 1.

12. A method according to claim 11 for manufacturing an orthodontic, endodontic or dental implant or device.

13. A method according to claim 12 for manufacturing an endodontic instrument which is adapted for use in performing root canal therapy.

14. A method according to claim 13 for manufacturing an endodontic file.

15. A method of manufacturing an endodontic file instrument comprising:
(a) providing a precursor blank having a proximal end and a distal end;
(b) forming the precursor blank into a file that is sized for insertion into a root canal, and that is adapted for abrasive action in the root canal, the precursor blank comprising at least one component at least partially fabricated from an alloy of claim 1.

16. Use of an implant or device according to claim 1.

17. Use of an implant or device according to claim 4.

18. Use of a device according to claim 5.

19. Use of a device according to claim 6.

20. Use of a kit according to claim 7.

21. Use of a kit according to claim 8.

22. Use of a kit according to claim 9.

23. Use of a kit according to claim 10.

24. An article of manufacture comprising packaging material and a medical implant or device wherein said packaging material comprises a label which indicates that said medical implant or device can be used for the use of claim 16, and wherein said medical implant or device comprises at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, said alloy comprising an amorphous phase as a volume fraction of at least 50%.

25. An article of manufacture comprising packaging material and a medical implant or device wherein said packaging material comprises a label which indicates that said medical implant or device can be used for the use of claim 17, and wherein said medical implant or device comprises at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, said alloy comprising an amorphous phase as a volume fraction of at least 50%.

26. An article of manufacture comprising packaging material and a medical device wherein said packaging material comprises a label which indicates that said medical device can be used for the use of claim 18, and wherein said medical implant or device comprises at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, said alloy comprising an amorphous phase as a volume fraction of at least 50%.

27. An article of manufacture comprising packaging material and a medical device wherein said packaging material comprises a label which indicates that said medical device can be used for the use of claim 19, and wherein said medical implant or device comprises at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, said alloy comprising an amorphous phase as a volume fraction of at least 50%.

28. An article of manufacture comprising packaging material and a kit wherein said packaging material comprises a label which indicates that said kit can be used for the use of claim 20, and wherein said kit comprises a plurality of medical implants and/or devices, said medical implants and/or devices comprising at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, said alloy comprising an amorphous phase as a volume fraction of at least 50%.

29. An article of manufacture comprising packaging material and a kit wherein said packaging material comprises a label which indicates that said kit can be used for the use of claim 21, and wherein said kit comprises a plurality of medical implants and/or devices, said medical implants and/or devices comprising at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, said alloy comprising an amorphous phase as a volume fraction of at least 50%.

30. An article of manufacture comprising packaging material and a kit wherein said packaging material comprises a label which indicates that said kit can be used for the use of claim 22, and wherein said kit comprises a plurality of medical implants and/or devices, said medical implants and/or devices comprising at least one component at least partially fabricated from an alloy having a composition represented by the general formula Xₐ M_{b} Al_{c}, where X is Zr or Hf, M is at least one element selected from the group consisting of Mn, Fe, Co, Ni, Ti and Cu, and a, b and c are, in atomic percentages, 25<a<85, 5<b<70 and 0<c<35, said alloy comprising an amorphous phase as a volume fraction of at least 50%.
